(19) United States
(12) Patent Application Publication
(10) Pub. No.: US 2012/0128641 A1
(43) Pub. Date: May 24, 2012

(54) METHODS AND COMPOSITIONS FOR IMPROVING THE VIABILITY OF CRYOPRESERVED CELLS

(75) Inventor: William G. Austen, JR., Weston, MA (US)

(73) Assignee: The General Hospital Corporation d/b/a Massachusetts General Hospital, Boston, MA (US)

(21) Appl. No.: 13/386,073

(22) PCT Filed: Jul. 20, 2010
(86) PCT No.: PCT/US10/02033

§ 371 Date: Feb. 13, 2012

Related U.S. Application Data
(60) Provisional application No. 61/227,023, filed on Jul. 20, 2009.

(51) Int. Cl. A61K 35/12; C12N 5/071; C12N 5/077; C12N 5/02

(52) U.S. Cl. 424/93.7; 435/374; 435/325; 435/366

(57) ABSTRACT

The present invention provides polymers and methods for increasing the viability of cryopreserved cells after thawing. Thawing cryopreserved cells in the presence of a polymer such as poloxymer P1 88 or other non-ionic polymers is thought to stabilize the membranes of the cells leading to increased post-thaw viability. Such methods may be used in the processing of cells and tissues for transplantation or for research purposes. Other agents such as antioxidants, vitamins, or osmotic protectants may also be added to cells to improve viability.

### RELATED APPLICATIONS

The present application claims priority under 35 U.S.C. §119(e) to U.S. provisional patent application, U.S. Ser. No. 61/227,023, filed Jul. 20, 2009, which is incorporated herein by reference.

### FIELD OF THE INVENTION

The present invention relates to polymers and methods for improving the viability of cryopreserved cells, which are particularly useful in the processing of cells and tissues for transplantation.

### BACKGROUND OF THE INVENTION

Cryopreservation is a process by which cells or tissues are preserved by cooling to sub-zero temperatures, such as by storage in liquid nitrogen. An ongoing problem with cryopreservation is that cells being preserved are often damaged due to solution concentration effects, ice formation, and dehydration, which can result in low cell viability post-thaw. Although many of these effects can be reduced by cryoprotectants, cryopreservation currently is limited by the toxicity of standard cryoprotective agents such as DMSO. For certain applications, such as clinical transplantion applications, standard cryoprotective agents are often unsuitable. Thus, there remains a need for identifying new methods and agents for cryoprotection. In particular, improved methods for the cyropreservation of fat would greatly enhance reconstruction with fat grafts by allowing for multiple treatments without additional harvesting.

### SUMMARY OF THE INVENTION

The present invention stems from the recognition that certain polymers improve the viability of cryopreserved cells when added during the process of thawing the cells. In particular embodiments, the polymers improve viability of cryopreserved cells irrespective of the use of a cryoprotective agent, e.g., DMSO, Trehalose, sucrose, glycerol, etc., during freezing. Preventing damage to the cryopreserved cells allows for the more successful and predictable recovery of cells for downstream applications, e.g., for clinical transplantation, cell-based drug screening, cell biological research, etc. Successful cryopreservation also reduces the need to repeat harvesting of cells. In certain embodiments, the polymers which improve the viability of cryopreserved cells are non-toxic or have reduced toxicity compared with cryoprotectants known in the art. Accordingly, in some embodiments, the polymers and methods disclosed herein are particularly useful for downstream clinical applications. In some embodiments, the present invention provides compositions that seal and/or stabilize the membrane of cryopreserved cells, e.g., post-thaw, and, consequently, improve the viability of cryopreserved cells post-thaw. Typically such compositions include a non-ionic polymer, e.g., a non-ionic polyether, that interacts with the phospholipid bilayer of a cell. The invention also provides methods of using such compositions in the processing and transplantation of tissues and cells (e.g., fat cells, stem cells, etc.).

In one aspect, the invention utilizes polymers that aid in increasing the viability of cryopreserved cells post-thaw. The viability of cryopreserved cells post-thaw may be evaluated using methods known in the art, including, for example, glycerol-3-phosphate dehydrogenase (G3PH) activity assays, ATP level assays, cell count assays, apoptotic activity assays, histology, DNA content, etc. Without wishing to be bound by a particular theory, the polymers may act to seal and/or stabilize the membranes of cells following cryopreservation. Any polymer may be used that seals or stabilizes the membrane of a cryopreserved cell when used during thawing of the cells. Preferably, the polymer utilized in the present invention is biocompatible and/or biodegradable. In some embodiments, the polymer is a non-ionic polymer. In certain embodiments, the polymer is a polyether. In certain embodiments, the polyether is a polyalkylether. In certain embodiments, the polyether is a block co-polymer of a polyalkylether and another polymer (e.g., a polyalkylether). In particular, poloxymers (also known as poloxamers) are disclosed herein as being useful in sealing and stabilizing cell membranes following cryopreservation. As shown in the chemical structure below, poloxymers are non-ionic triblock copolymers composed of a central hydrophobic chain of polyoxypropylene (also known as polypropylene glycol) flanked by two hydrophilic chains of polyoxyethylene (also known as polyethylene glycol).

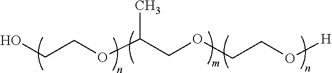

In certain embodiments, poloxymer P188 is used to increase the viability of cells, e.g., cells of a fat graft, following cryopreservation. Poloxamers are sold by BASF under the trade name PLURONIC®. In particular, poloxamer 188 (P188) is sold under the tradename PLURONIC® F68. Since the lengths of the blocks making up the polymer can be customized, many different poloxamers with different properties exist. These copolymers are commonly named with the letter “P” for poloxamer followed by three digits. The first two digits×100 give the approximate molecular weight of the hydrophobic polyoxypropylene core, and the last digit×10 gives the percentage of polyoxyethylene content. Poloxamer 188 is a poloxymer with a polyoxypropylene molecular mass of 1800 g/mol and an 80% polyoxyethylene content, and therefore, poloxamer 188 has an average molecular weight of 7680-9510 g/mol. To convert the “Pxxy” name to the tradename “Fzz”, the xx of “Pxxy” is multiplied by approximately 3, that is, P188 is F68. Other poloxymers that may be useful in the present invention include poloxamers P108 (PLURONIC® F38), P184 (PLURONIC® L64), P401, P402, P407 (PLURONIC® F127), and P408 (PLURONIC® F108). Other poloxamers with a lower molecular weight and approximately equal or lower PEG content may be useful in the present invention. Other particular polymers that may be useful in increasing the viability of cryopreserved cells post-thaw include polyethylene glycol (PEG), polysorbate 80, certain TETRONIC® surfactants, meroxapols, poloxamines (e.g., 304, 701, 704, 901, 904, 908, 1307), and PLURADOT™ polyols. The polymer, e.g., the polyether, is added to the cryopreserved cells prior to thawing, immediately prior to thawing, after beginning thawing, immediately after the thawing, or during the freezing. Typically, the polymer is added to the cryopreserved cells at a concentration ranging from approximately 1 mg to approximately 20 mg of polymer per ml of cells. In certain embodiments, P188 is at a concentration of approximately 10 mg/ml. In certain embodiments, a millimolar concentration of the polymer is used. Typically the lowest concentration of polymer that yields the desired membrane stabilization following cryopreservation is used. As would be appreciated by one of skill in the art, the concentration of polymer in the composition will depend on the polymer being used to stabilize, e.g., increase viability of the cryopreserved cells, the type of cryopreserved cells, the cryoprotectant used, the thaw process, the ultimate use of the cells, etc.

In some aspects of the invention, cryopreserved cells are thawed in the presence of a polymer, e.g., a polyether. Typically, the cells to be transplanted are thawed in the presence of the polymer at an appropriate concentration and are then transplanted into the recipient (e.g., a human) at a desired transplant site (e.g., face, lips). The thawed cells may be washed to remove any excess polymer before transplantation. Any cryopreserved cells may be thawed and transplanted using the inventive technology. In certain embodiments, the cells are derived from fat tissue. In certain embodiments, the cells are adipocytes. In certain embodiments, the cells are fibroblasts. In some embodiments, the cells are mammalian cells, e.g., human cells. In certain embodiments, the cells are cord-blood cells, stem cells, embryonic stem cells, adult stem cells, cancer stem cells, progenitor cells, autologous cells, isograft cells, allograft cells, xenograft cells, cell lines, or genetically engineered cells. The polymer may be mixed with the cryopreserved cells before thawing, e.g., immediately before thawing, or after thawing has begun. The polymer may be mixed with cells immediately following thawing. In some embodiments, thawed cells may be mixed with the polymer just prior to transplantation. The cell/polymer composition may also include other agents. For example, the composition may include agents that further protect or stabilize the cells to be transplanted, or the agent may protect the polymer. In certain embodiments, the composition includes vitamins, minerals, antioxidants, reductants, osmotic protectants, viscosity enhancers, coenzymes, membrane stabilizers, lipids, carbohydrates, hormones, growth factors, anti-inflammatory agents, polynucleotides, proteins, peptides, alcohols, organic acids, small organic molecules, etc.

In another aspect, the invention provides kits useful in transplanting cryopreserved cells or tissues using the inventive compositions and methods. The kit may include all or a subset of all the components necessary for transplanting cryopreserved cells or tissues, e.g., fat-derived cells or fat tissue, into a subject. The kits may include, for example, polymer, cells, syringe, needle, containers, alcohol swabs, anesthetics, wash solution, antibiotics, antiseptics, antioxidants, vitamins, lipids, carbohydrates, hormones, growth factors, etc. In certain embodiments, the cells are acquired from the patient to receive the cells (i.e., an autologous graft). In certain embodiments, the components of the kit are sterilely packaged for convenient use by the surgeon or other health care professional. The kit may also include instructions for using the polymer and other agents in the thawing process or transplantation procedure. The kit may provide the necessary components for a single use. The kit may also include packaging and information as required by a governmental regulatory agency that regulates pharmaceuticals and/or medical devices.

### BRIEF DESCRIPTION OF THE DRAWING

FIG. 1 depicts a viability assessment of explanted fat nodules which were weighed and analyzed for glycerol-3-phosphate dehydrogenase (G3PH) activity, ATP levels, cell counts, and apoptotic activity.

FIG. 2 depicts histological images of H&E staining of samples following 6 weeks cryopreservation and 6 weeks in vivo implantation into nude mice. Both saline and DMSO+Trehalose groups demonstrate significant areas of fibrotic reaction and inflammatory infiltrate. P188-treated samples and P188 plus DMSO/Trehalose samples demonstrate significantly lower amounts of fibrosis and infiltrate, and appear most similar to histological images of H&E staining of fresh fat graft.

FIG. 3 depicts the effectiveness of thawing cryopreserved cells in the presence of P188 for reducing the amount of post-thaw cell death.

FIG. 4 depicts functional improvements in fat grafts which have been thawed in the presence of P188.

FIG. 5 shows a comparison of the weights of fat grafts treated with normal saline (NS), P188, and DMSO+Trehalose (DMT) 6 weeks post-implantation. P188 demonstrated statistically significant differences in reabsorption.

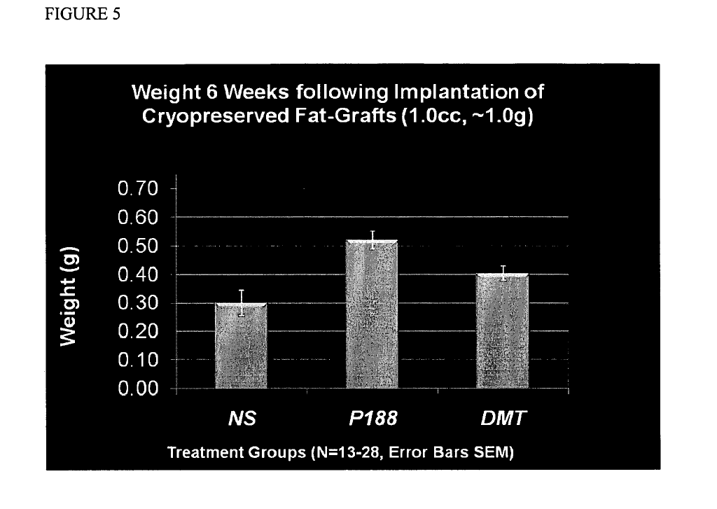

FIG. 6 shows the viability of fat grafts treated with normal saline (NS), P188, and DMSO+Trehalose (DMT) 6 weeks post-implantation. At 6 weeks, P188 demonstrated statistically significant differences (p<0.05) in live cell signal.

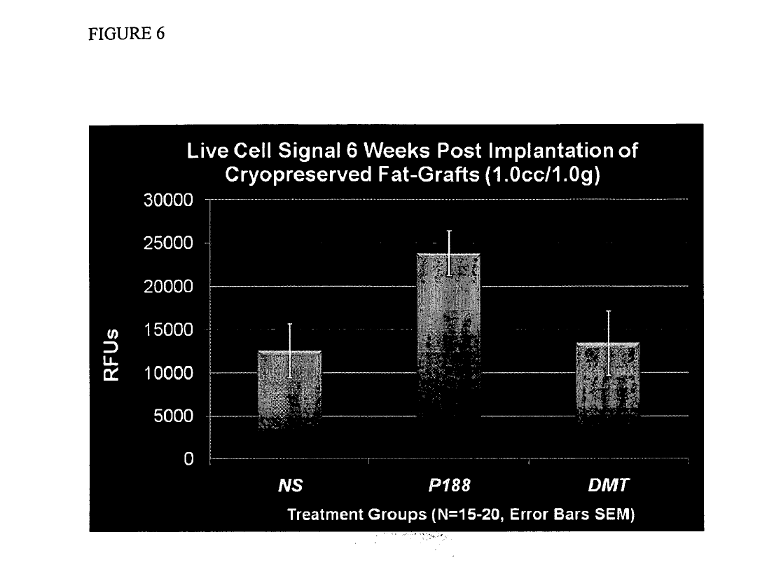

FIG. 7 shows the DNA content of fat grafts treated with normal saline (NS), P188, and DMSO+Trehalose (DMT) 6 weeks post-implantation.

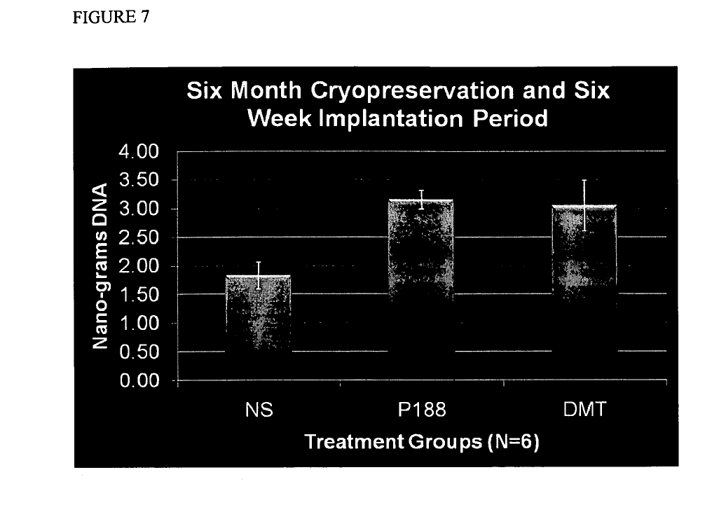

FIG. 8 is a comparison of P188 as a thaw treatment versus a pre-treatment. (A) Weight of fat grafts 6 weeks post-implantation. (B) Viability 6 weeks post-implantation.

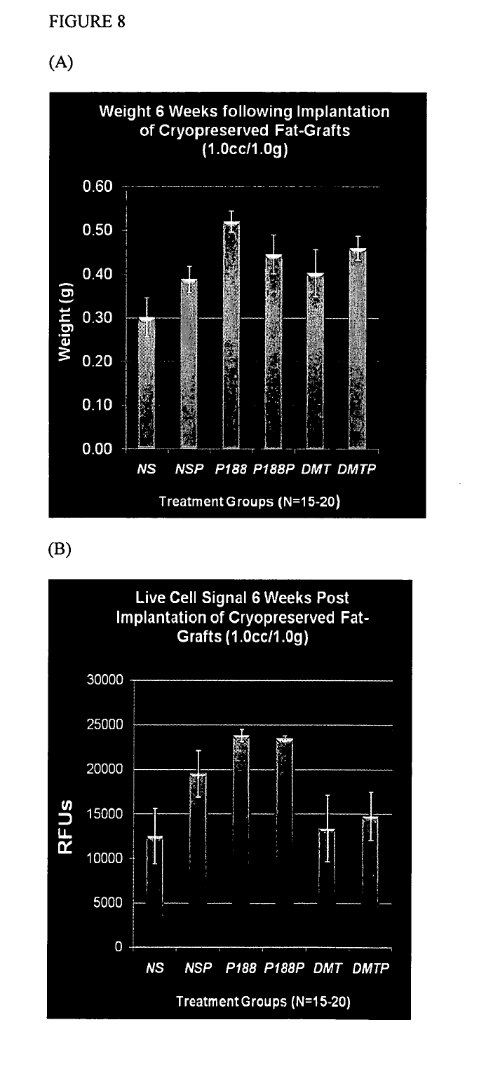

### DEFINITIONS

“Anti-inflammatory agent,” as used herein, refers to any substance that inhibits one or more signs or symptoms of inflammation.

The term “approximately” in reference to a number generally includes numbers that fall within a range of 5% in either direction of the number (greater than or less than the number) unless otherwise stated or otherwise evident from the context (except where such number would exceed 100% of a possible value).

“Polyethers” are compounds with more than one ether group. An ether group has an oxygen atom connected to two (substituted) alkyl or aryl groups of general formula R—O—R′. Polyethers may be homopolymers or co-polymers. Polyethers may be block co-polymers, such as diblock, triblock, and tetrablock copolymers.

“Cryopreserved cells” are cells that have been preserved by cooling to a sub-zero temperature. Cryopreserved cells may or may not be preserved in the presence of a cryoprotective agent. A cryoprotective agent is a substance that protects cells from damage associated with storage at sub-zero temperature and/or freezing, e.g., cell membrane damage due to ice crystal formation. Cryopreserved cells include eukaryotic and prokaryotic cells. Cryopreserved cells include animal and plant cells.

“Biocompatible” refers to a material that is substantially nontoxic to cells in the quantities used, and also does not elicit or cause a significant deleterious or untoward effect on the recipient's body at the location used, e.g., an unacceptable immunological or inflammatory reaction, unacceptable scar tissue formation, etc.

“Biodegradable” means that a material is capable of being broken down physically and/or chemically within cells or within the body of a subject, e.g., by hydrolysis under physiological conditions and/or by natural biological processes such as the action of enzymes present within cells or within the body, and/or by processes such as dissolution, dispersion, etc., to form smaller chemical species which can typically be metabolized and, optionally, used by the body, and/or excreted or otherwise disposed of. For purposes of the present invention, a polymer whose molecular weight decreases over time in vivo due to a reduction in the number of monomers is considered biodegradable.

The terms “polynucleotide”, “nucleic acid”, or “oligonucleotide” refer to a polymer of nucleotides. The terms “polynucleotide”, “nucleic acid”, and “oligonucleotide”, may be used interchangeably. Typically, a polynucleotide comprises at least two nucleotides. DNAs and RNAs are polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, 2′-methoxyribose, 2′-aminoribose, ribose, 2′-deoxyribose, arabinose, and hexose), or modified phosphate groups (e.g., phosphorothioates and 5′-N phosphoramidite linkages). Enantiomers of natural or modified nucleosides may also be used. Nucleic acids also include nucleic acid-based therapeutic agents, for example, nucleic acid ligands, siRNA, short hairpin RNA, antisense oligonucleotides, ribozymes, aptamers, and SPIEGELMERS™, oligonucleotide ligands described in Wlotzka, et al., Proc. Natl. Acad. Sci. USA, 2002, 99(13):8898, the entire contents of which are incorporated herein by reference.

A “polypeptide”, “peptide”, or “protein” comprises a string of at least three amino acids linked together by peptide bonds. The terms “polypeptide”, “peptide”, and “protein”, may be used interchangeably. Peptide may refer to an individual peptide or a collection of peptides. Inventive peptides preferably contain only natural amino acids, although non natural amino acids (i.e., compounds that do not occur in nature but that can be incorporated into a polypeptide chain) and/or amino acid analogs as are known in the art may alternatively be employed. Also, one or more of the amino acids in a peptide may be modified, for example, by the addition of a chemical entity such as a carbohydrate group, a phosphate group, a farnesyl group, an isofarnesyl group, a fatty acid group, a linker for conjugation, functionalization, or other modification, etc. In one embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

The terms “polysaccharide” and “carbohydrate” may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replace with a fluorine, or N-acetylglucosamine, a nitrogen-containing form of glucose. (e.g., 2′-fluororibose, deoxyribose, and hexose). Carbohydrates may exist in many different forms, for example, conformers, cyclic forms, acyclic forms, stereoisomers, tautomers, anomers, and isomers.

“Small molecule” refers to organic compounds, whether naturally-occurring or artificially created (e.g., via chemical synthesis) that have relatively low molecular weight and that are not proteins, polypeptides, or nucleic acids. Small molecules are typically not polymers with repeating units. In certain embodiments, a small molecule has a molecular weight of less than about 1500 g/mol. In certain embodiments, the molecular weight of the polymer is less than about 1000 g/mol. Also, small molecules typically have multiple carbon-carbon bonds and may have multiple stereocenters and functional groups.

“Subject,” as used herein, refers to an individual to whom an agent is to be delivered, e.g., for experimental, diagnostic, and/or therapeutic purposes. Preferred subjects are mammals, particularly domesticated mammals (e.g., dogs, cats, etc.), primates, or humans. In certain embodiments, the subject is a human. In certain embodiments, the subject is an experimental animal such as a mouse or rat. A subject under the care of a physician or other health care provider may be referred to as a “patient.”

“Pharmaceutical agent,” also referred to as a “drug,” is used herein to refer to an agent that is administered to a subject to treat a disease, disorder, or other clinically recognized condition that is harmful to the subject, or for prophylactic purposes, and has a clinically significant effect on the body to treat or prevent the disease, disorder, or condition. Therapeutic agents include, without limitation, agents listed in the United States Pharmacopeia (USP), Goodman and Gilman's The Pharmacological Basis of Therapeutics, 10^{th} Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17^{th} ed. (1999), or the 18^{th} ed (2006) following its publication, Mark H. Beers and Robert Berkow (eds.), Merck Publishing Group, or, in the case of animals, The Merck Veterinary Manual, 9^{th} ed., Kahn, C. A. (ed.), Merck Publishing Group, 2005.

### DETAILED DESCRIPTION OF CERTAIN EMBODIMENTS OF THE INVENTION

The present invention stems from the recognition that certain polymers, e.g., polyethers, improve the viability of cryopreserved cells when added before or during the process of thawing the frozen cells. The improvement in viability is typically observed irrespective of the use of the cryoprotective agent added to the cells prior to freezing. Without wishing to be bound by a particular theory, the polymer is thought to interact with the cell membranes and seal or prevent defects in the cellular membranes during the process of thawing, or immediately following the thawing, thereby preventing or minimizing injury to the cell once thawed. Preventing injury to the cryopreserved cells reduces the extent of apoptosis and cell death after thawing and aids in improving the success and consistency of certain downstream applications, particularly downstream clinical applications such as transplantion. In certain embodiments, the present invention provides polymers, compositions, and methods for improving fat transplantation in a subject (e.g., humans). The inventive system may also be used in storing/cryopreserving other types of cells including stem cells.

### Polymers

The present invention is based on the discovery of polymers that aid in sealing and/or stabilizing the membranes of cells following cryopreservation and methods for accomplishing the same. The polymer is mixed with the cryopreserved cells, e.g., prior to thawing, during thawing, etc., at a sufficient concentration to stabilize and protect the membranes of the cells from damage post-thaw. Such polymers may be used in conjunction with other techniques and materials for improving the success of downstream applications, such as cell transplantation.

Any polymer may be used that seals or stabilizes the membrane of a cryopreserved cell when added during thawing of the cells. In certain embodiments, the polymer is a synthetic polymer (i.e., a polymer not produced in nature). In certain embodiments, the polymer is a surface active polymer. The polymer may be a homopolymer, a copolymer, a block copolymer, a branched polymer, a dendritic polymer, a star polymer, a blend of polymers, a cross-linked polymer, or an uncross-linked polymer. In certain embodiments, the polymer is a non-ionic polymer. In certain embodiments, the polymer is a non-ionic block copolymer. In certain embodiments, the polymer is a non-ionic tri-block copolymer.

In particular embodiments, the polymer is a polyether. In certain embodiments, the polyether is a polyalkylether. In certain embodiments, the polyether is polyethylene glycol. In certain embodiments, the polyether is polypropylene glycol. In certain embodiments, the polyether is polybutylene glycol. In certain embodiments, the polyether is polypentylene glycol. In certain embodiments, the polyether is polyhexylene glycol. In certain embodiments, the polymer is a block copolymer of one of the above-mentioned polymers.

In certain embodiments, the polyether is block copolymer of a polyalkyl ether (e.g., polyethylene glycol, polypropylene glycol) and another polymer. In certain embodiments, the polyether is a block copolymer of a polyalkyl ether and another polyalkyl ether. In certain embodiments, the polyether is a block copolymer of polyethylene glycol and another polyalkyl ether. In certain embodiments, the polyether is a block copolymer of polypropylene glycol and another polyalkyl ether. In certain embodiments, the polyether is a block copolymer with at least one unit of polyalkyl ether. In certain embodiments, the polyether is a block copolymer of two different polyalkyl ethers. In certain embodiments, the polyether is a block copolymer including a polyethylene glycol unit. In certain embodiments, the polyether is a block copolymer including a polypropylene glycol unit. In certain embodiments, the polyether is a tri-block copolymer of a more hydrophobic unit flanked by two more hydrophilic units. In certain embodiments, the polyether is a tri-block copolymer of a more hydrophilic unit flanked by two more hydrophobic units. In certain embodiments, the polyether includes a polypropylene glycol unit flanked by two more hydrophilic units. In certain embodiments, the polyether includes two polyethylene glycol units flanking a more hydrophobic unit. In certain embodiments, the polyether is a tri-block copolymer with a polyproylene glycol unit flanked by two polyethylene glycol units. In certain embodiments, the polyether is of the formula:

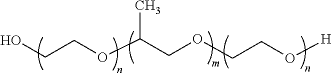

wherein n is an integer between 2 and 200, inclusive; and m is an integer between 2 and 200, inclusive. In certain embodiments, n is an integer between 10 and 100, inclusive. In certain embodiments, m is an integer between 5 and 50 inclusive. In certain embodiments, n is approximately 2 times m. In certain embodiments, n is approximately 70, and m is approximately 35. In certain embodiments, n is approximately 50, and m is approximately 30. In certain embodiments, the polymer is poloxamer P188, which is marketed by BASF under the trade name PLURONIC® F68. Other PLURONIC® polymers that may be useful in the present invention include, but are not limited to, PLURONIC® 10R5, PLURONIC® 17R2, PLURONIC® 17R4, PLURONIC® 25R2, PLURONIC® 25R4, PLURONIC® 31R1, PLURONIC® 10R5, PLURONIC® F108, PLURONIC® F127, PLURONIC® F38, PLURONIC® F68, PLURONIC® F77, PLURONIC® F87, PLURONIC® F88, PLURONIC® F98, PLURONIC® L10, PLURONIC® L101, PLURONIC® L121, PLURONIC® L31, PLURONIC® L35, PLURONIC® L43, PLURONIC® L44, PLURONIC® L61, PLURONIC® L62, PLURONIC® L64, PLURONIC® L81, PLURONIC® L92, PLURONIC® N3, PLURONIC® P103, PLURONIC® P104, PLURONIC® P105, PLURONIC® P123, PLURONIC® P65, PLURONIC® P84, and PLURONIC® P85. Poloxamers are generally synthesized by the sequential addition of first propylene oxide and then ethylene oxide to propylene glycol.

In certain embodiments, the polyether is a di-block copolymer. In certain embodiments, the polyether is a tetra-block copolymer. In certain embodiments, the di-block or tetra-block copolymer includes a polyalkylether unit. In certain embodiments, the di-block or tetra-block copolymer includes a polypropylene glycol unit. In certain embodiments, the di-block or tetra-block copolymer includes a polyethylene glycol unit. In certain embodiments, the polyether is a tetra-block copolymer of polyethylene glycol and polypropylene glycol unites. In certain embodiments, the tetra-block copolymer is a TETRONIC® polymer marketed by BASF. Exemplary TETRONIC® polymers include TETRONIC® 1301. TETRONIC® 1304, TETRONIC® 1307, TETRONIC® 150R1, TETRONIC® 304, TETRONIC® 701, TETRONIC® 901, TETRONIC® 904, TETRONIC® 908, and TETRONIC® 90R4. In certain embodiments, the polyether is a block copolymer of more than four block units.

In certain embodiments, the polyether is a meroxapol. Meroxapols are prepared when the order of addition of the alkylene oxide is reversed. That is, ethylene oxide is added first to a polyethylene glycol core followed by propylene glycol. The hydrophilic portion is flanked by two more hydrophobic units. In certain embodiments, the polyether is a poloxamine. Poloxamines are block copolymers which have a tetrafunctional structure of four polyethyleneoxide/polypropyleneoxide units centered on an ethylenediamine core. Exemplary poloxamines include, but are not limited to, poloxamine 304, 504, 701, 704, 901, 904, 908, 1101, 1102, 1302, 1304, 1307, 1501, 1504, and 1508. In certain embodiments, the polyether is a PLURADOT™ polyol. See Schmolka, “A Review of Block Polymer Surfactants” J. Am. Oil Chemists's Soc. 54(3):110-116, 1977; incorporated herein by reference.

The molecular weight of the polyether utilized in the present invention may range from approximately 500 g/mol up to approximately 50,000 g/mol. In certain embodiments, the molecular weight of the polyether ranges from approximately 1,000 g/mol to approximately 30,000 g/mol. In certain embodiments, the molecular weight of the polyether ranges from approximately 2,000 g/mol to approximately 15,000 g/mol. In certain embodiments, the molecular weight of the polyether ranges from approximately 2,000 g/mol to approximately 12,000 g/mol. In certain embodiments, the molecular weight of the polyether ranges from approximately 1,000 g/mol to approximately 5,000 g/mol. In certain embodiments, the molecular weight of the polyether ranges from approximately 5,000 g/mol to approximately 10,000 g/mol. In certain embodiments, the molecular weight of the polyether ranges from approximately 10,000 g/mol to approximately 15,000 g/mol. In certain embodiments, the molecular weight of the polyether ranges from approximately 15,000 g/mol to approximately 20,000 g/mol. In certain embodiments, the molecular weight of the polyether is approximately 20,000 g/mol to approximately 25,000 g/mol. In certain embodiments, the average molecular weight of P188 is approximately 8,400 g/mol. The average molecular weight of other commercially available poloxamers are known in the art.

The composition of polyether used in the present invention is typically pharmaceutical grade material for use in humans and/or other animals. In certain embodiments, the polyether is approved for use in humans and for veterinary use. In some embodiments, the polyether is approved by for use in humans by the United States Food and Drug Administration. In some embodiments, the polyether is pharmaceutical grade material. In some embodiments, the polyether meets the standards of the United States Pharmacopoeia (USP), the European Pharmacopoeia (EP), the British Pharmacopoeia, and/or the International Pharmacopoeia. In certain embodiments, the polyether is at least 90% pure. In certain embodiments, the polyether is at least 95% pure. In certain embodiments, the polyether is at least 98% pure. In certain embodiments, the polyether is at least 99% pure. In certain embodiments, the polyether is at least 99.5% pure. In certain embodiments, the polyether is at least 99.9% pure. In certain embodiments, the polyether is at least 99.99% pure. In certain embodiments, the polyether is free of toxic or non-biocompatible materials.

The polyether useful in the present invention typically degrades in vivo into non-toxic degradation products or is safely excreted by the body. The polymer is preferably biocompatible and does not result in any substantial unwanted side effects. The polymer's half-life in vivo can range from approximately 1 day to approximately 1 month. In certain embodiments, the half-life of the polyether in vivo ranges from approximately 1 day to approximately 1 week. In certain embodiments, the half-life of the polyether in vivo ranges from approximately 1 week to approximately 2 weeks. In certain embodiments, the half-life of the polyether in vivo ranges from approximately 3 weeks to approximately 4 weeks.

### Uses

The polymers utilized in the present invention are useful for improving the viability of cryopreserved cells. The methods typically involve thawing cryopreserved cells in the presence of a polymer, e.g., a polyether such as P188. Methods are provided for processing cells that involve cryopreserving cells and thawing the cryopreserved cells in the presence of a polymer. The cells may be optionally washed at any stage (e.g., after harvesting, before freezing, after thawing, or before transplantation). The polymer may be added to the cells prior to freezing. The polymer may be added with a cryoprotectant, e.g., before the cells are frozen. The polymer may be added to the cryopreserved cells before thawing. For example, cryopreserved cells, e.g., which have been frozen in the absence of the polymer, may be removed from storage in a frozen state, the polymer may be added to the cells, and the cells may be returned to a freezer with the polymer present for thawing. The polymer may also be added to the cryopreserved cells immediately before thawing. For example, cryopreserved cells may be removed from storage in a frozen state and the polymer may be immediately added to the cells, e.g., before placing the cells in an incubation chamber (e.g., water bath, heat block, oven), such that the cells are thawed in the presence of the polymer. The polymer may also be added to the cryopreserved cells after thawing has begun, e.g., after placing cells in an incubation chamber. The polymer may also be added before cryopreserving the cells. The polymer may also be added after the cryopreserved cells are thawed. The polymer may be added at any stage—before, during, or after the freezing or thawing of the cells.

In view of the teachings provided herein and known in the art, the skilled artisan will be capable of controlling the addition of the polymer to maximize the viability of the cryopreserved cells following thawing. Methods for thawing cryopreserved cells are well known in the art (See, e.g., Freshney R I, Culture of Animal Cells: A Manual of Basic Technique, 4^{th} Edition, 2000, Wiley-Liss, Inc., Chapter 19). The polymers disclosed herein that improve post-thaw viability are amenable to use with such art known methods.

It will be appreciated that the thawing rate of cryopreserved cells will be influenced by a variety of factors. For example, the volume of the cryopreserved cells, handling time, ambient temperature, temperature of incubation chambers used, heat transfer properties of the container housing the cells, the volume of the polymer added to the cryopreserved cells, and the temperature of the polymer added to the cryopreserved cells may influence thawing rate. It will also be appreciated that cells in a particular sample of cryopreserved cells may not all thaw at the same rate or within the same time period. Thus, polymer added to a sample of cryopreserved cells may contact some cells after thawing and other cells during the thawing, depending on the timing of addition of the polymer to the cryopreserved cells and other factors disclosed herein and apparent to the skilled artisan.

The cryopreserved cells to be thawed in the presence of a polymer may be in a composition that occupies a volume of up to about 1 ml, about 2 ml, about 3 ml, about 4 ml, about 5 ml, about 10 ml, about 20 ml, about 30 ml, about 40 ml, about 50 ml, about 100 ml, about 200 ml, about 300 ml, about 400 ml, about 500 ml, about 1 L, or more. The cryopreserved cells may be in a composition that occupies a volume ranging from about 1 ml to about 10 ml, from about 10 ml to about 20 ml, from about 20 ml to about 30 ml, from about 30 ml to about 40 ml, from about 40 ml to about 50 ml, from about 50 ml to about 100 ml, from about 100 ml to about 200 ml, from about 200 ml to about 300 ml, from about 300 ml to about 400 ml, from about 400 ml to about 500 ml, or from about 500 ml to about 1 L. The composition comprising the cells may be a tissue, e.g., a blood sample, a fat sample. The composition comprising the cells may further comprise other agents, e.g., cryoprotective agents such as glycerol DMSO, sucrose, or Trehalose.

Typically, the step of thawing involves obtaining cryopreserved cells from storage at a temperature of less than about 0° C. (a subzero temperature) and allowing them to come to a temperature above 0° C. The step of thawing may involve obtaining the cryopreserved cells from storage at a temperature that ranges from about −205° C. to about −195° C. The step of thawing may involve obtaining the cryopreserved cells from storage at a temperature that ranges from about −80° C. to about −60° C. The step of thawing may involve progressively warming the cryopreserved cells by transferring the cells among incubators each have a warmer temperature range, e.g., to control the rate of thawing. For example, the step of thawing may involve first obtaining cryopreserved cells from storage at a first subzero temperature, e.g., that ranges from about −205° C. to about −195° C., and transferring the cryoperserved cells to a second, typically warmer, yet typically subzero, storage temperature, e.g., to a temperature that ranges from about −80° C. to about −60° C., prior to thawing. Any number of stages, e.g., 2, 3, 4, 5, 6, or more stages, are envisioned to control the rate of thawing in this manner. The step of thawing may also involve progressively warming the cryopreserved cells by incubating the cells in a temperature controlled chamber, e.g., a water bath, heat block, oven, etc., and progressively warming the chamber, e.g., at a controlled rate, while the cryopreserved cells are present in the chamber.

The step of thawing may involve incubating the cryopreserved cells at a temperature that ranges from about 15° C. to about 30° C. The step of thawing may involve incubating the cryopreserved cells at a temperature that ranges from about 30° C. to about 45° C. Such incubation may be performed by incubating a container housing the cryoperserved cells in temperature controlled incubator, e.g., a temperature controlled water bath, a temperature controlled oven, etc. Other incubation methods will be apparent to the skilled artisan.

The step of thawing may be completed within about 30 seconds, about 1 minute, about 2 minutes, about 3 minutes, about 4 minutes, about 5 minutes, about 10 minutes, about 20 minutes, about 30 minutes, about 40 minutes, about 50 minutes, about 1 hour, or more. The step of thawing may be completed within a range of about 1 minute to about 5 minutes. The step of thawing may be completed within a range of about 5 minutes to about 10 minutes. The step of thawing may be completed within a range of about 10 minutes to about 30 minutes. The step of thawing may be completed within a range of about 30 minutes to about 60 minutes.

The step of thawing may involve warming the cryopreserved cells at a rate of about 1° C. per minute, about 2° C. per minute, about 3° C. per minute, about 4° C. per minute, about 5° C. per minute, about 10° C. per minute, about 20° C. per minute, about 30° C. per minute, about 40° C. per minute, about 50° C. per minute, about 60° C. per minute, about 70° C. per minute, about 80° C. per minute, about 90° C. per minute, about 100° C. per minute, about 200° C. per minute, or more. The step of thawing may involve warming the cryopreserved cells at a rate ranging from about 1° C. per minute to about 5° C. per minute. The step of thawing may involve warming the cryopreserved cells at a rate ranging from about 5° C. per minute to about 25° C. per minute. The step of thawing may involve warming the cryopreserved cells at a rate ranging from about 25° C. per minute to about 50° C. per minute. The step of thawing may involve warming the cryopreserved cells at a rate ranging from about 50° C. per minute to about 100° C. per minute. The step of thawing may involve warming the cryopreserved cells at a rate ranging from about 100° C. per minute to about 200° C. per minute. The rate of thawing may be continuous, e.g., a constant rate until cells are completely thawed. The rate of thawing may also be discontinuous, e.g., the rate may be more rapid at some temperature ranges relative to the rate at other temperature ranges during thawing, e.g., the rate may be more rapid in the range of about −200° C. to about 0° C. then in the range of about 0° C. to about 45° C. during the thawing.

The cells may be frozen in the absence a cryopreservation agent. The cells may be frozen in the presence of one or more cryopreservation agents known in the art. In some embodiments, the cryopreservation agent is a simple or complex carbohydrate. In some embodiments, the cryopreservation agent is selected from the group consisting of an aldose, a ketose, an amino sugar, a disaccharide, a polysaccharide, and combinations thereof. In some embodiments, the cryopreservation agent is selected from the group consisting of sucrose, dextrose, glucose, lactose, trehalose, arabinose, pentose, ribose, xylose, galactose, hexose, idose, monnose, talose, heptose, fructose, gluconicacid, sorbitol, mannitol, methyl α-glucopyranoside, maltose, isoascorbic acid, ascorbic acid, lactone, sorbose, glucaric acid, erythrose, threose, arabinose, allose, altrose, gulose, erythrulose, ribulose, xylulose, psicose, tagatose, glucuronicacid, gluconic acid, glucaric acid, galacturonic acid, mannuronic acid, glucosamine, galactosamine, neuraminic acid, arabinans, fructans, fucans, galactans, galacturonans, glucans, mannans, xylans, levan, fucoidan, carrageenan, galactocarolose, pectins, pectic acids, amylose, pullulan, glycogen, amylopectin, cellulose, dextran, pustulan, chitin, agarose, keratin, chondroitin, dermatan, hyaluronic acid, alginic acid, xanthin gum, starch, polyethyleneglycol, dimethyl sulfoxide, ethylene glycol, propylene glycol, propylene, glycol, polyvinvyl pyrrolidone, glycerol, polyethylene oxide, polyether, serum, and combinations thereof. In certain embodiments, the cryopreservation agent is a poloxymer as described herein (e.g., P188).

The cryopreserved cells are typically mixed with the polymer during thawing at a concentration ranging from approximately 1-20 mg of polymer per mL of cells. As would be appreciated by one of skill in the art, the concentration of polymer needed to sufficiently stabilize the membranes of the cryopreserved cells and improve viability may vary depending on the polymer used, the subject, the cells, the concentration of the cells, the downstream application, e.g., transplantation, etc. In certain embodiments, the concentration ranges from approximately 1-10 mg of polymer per mL of cryopreserved cells. In certain embodiments, the concentration ranges from approximately 1-5 mg of polymer per mL of cryopreserved cells. In certain embodiments, the concentration ranges from approximately 5-10 mg of polymer per mL of cryopreserved cells. In certain embodiments, the concentration ranges from approximately 10-15 mg of polymer per mL of cryopreserved cells. In certain embodiments, the concentration ranges from approximately 15-20 mg of polymer per mL of cryopreserved cells. In certain embodiments, the concentration is approximately 5, 6, 7, 8, 9, 10, 11, 12, 13, 14, or 15 mg of polymer per mL of cryopreserved cells. In certain embodiments, when the poloxymer P188 is used, the concentration is approximately 5 mg of polymer per mL of cryopreserved cells (e.g., fat cells). In certain embodiments, when the poloxymer P188 is used, the concentration is approximately 8 mg of polymer per mL of cryopreserved cells (e.g., fat cells). In certain embodiments, when the poloxymer P188 is used, the concentration is approximately 10 mg of polymer per mL of cryopreserved cells (e.g., fat cells). In certain embodiments, when the poloxymer P188 is used, the concentration is approximately 12 mg of polymer per mL of cryopreserved cells (e.g., fat cells). In certain embodiments, when the poloxymer P188 is used, the concentration is approximately 15 mg of polymer per mL of cryopreserved cells (e.g., fat cells).

The cells may be washed at any stage during the cryopreservation process. In certain embodiments, the cells are washed after harvesting. In certain embodiments, the cells are washed after thawing. In certain embodiments, the cells are washed before transplantation. The cells may be washed after thawing to remove any excess polymer not associated with the cells. Such washing may prevent or minimize any adverse reaction to the polymer or any cellular debris from the cryopreservation process. The washing of cells may be performed using any known methods in the art. For example, the cells may be washed with normal saline or another suitable wash solution. In certain embodiments, the volume of wash solution used is at least equal to the volume of cells being washed. The washing may involve suspending the cells in the wash solution and then centrifuging the cells to collect the washed cells. In other embodiments, the cells are centrifuged without adding any wash solution, and the cell pellet is resuspended in normal saline or another suitable solution for further use such as transplantation. The step of washing may be performed once or multiple times. In certain embodiments, the wash step may be repeated two, three, four, five, six, seven, or more times. Typically, the wash step is not performed more than two to three times. In certain embodiments, only a single wash is performed.

The concentration of the cryopreserved cells may vary depending on a variety of factors, including for example the type of cell or tissue, the type of cryoprotectant used, the type of polymer used, the downstream application, etc. The concentration of certain cell types may be low, e.g., for oocytes the concentration may be as low as about 1-30 cells per ml, or lower. The concentration of cells may be about 10⁰ cells/ml, about 10¹ cells/ml, about 10² cells/ml, about 10³ cells/ml, about 10⁴ cells/ml, about 10⁵ cells/ml, about 10⁶ cells/ml, about 10⁷ cells/ml, about 10⁸ cells/ml, about 10⁹ cells/ml, or more. The concentration of cells may range from about 10⁰ cells/ml to about 10¹ cells/ml, from about 10¹ cells/ml to about 10² cells/ml, from about 10² cells/ml to about 10³ cells/ml, from about 10³ cells/ml to about 10⁴ cells/ml, from about 10⁴ cells/ml to about 10⁵ cells/ml, from about 10⁵ cells/ml to about 10⁶ cells/ml, from about 10⁶ cells/ml to about 10⁷ cells/ml, from about 10⁷ cells/ml to about 10⁸ cells/ml, or from about 10⁸ cells/ml to about 10⁹ cells/ml, for example.

The methods and compositions disclosed herein may be used with any cryopreserved cells, typically eukaryotic cells. However, the methods and compositions disclosed herein are also envisioned for use with prokaryotic cells. The methods and compositions disclosed herein are also useful with plant cells.

Cells may be primary cells isolated from any tissue or organ (e.g., connective, nervous, muscle, fat or epithelial tissue). The cells may be mesenchymal, ectodermal, or endodermal. Cells may also be present in isolated connective, nervous, muscle, fat or epithelial tissue, e.g., a tissue explant, e.g., an adipose tissue obtained by liposuction. The connective tissue may be, for example, bone, ligament, blood, cartilage, tendon, or adipose tissue. The muscle tissue may be vascular smooth muscle, heart smooth muscle, or skeletal muscle, for example. The epithelial tissue may be of the blood vessels, ducts of submandibular glands, attached gingiva, dorsum of tongue, hard palate, esophagus, pancrease, adrenal glands, pituitary glands, prostate, liver, thyroid, stomach, small intestine, large intestine, rectum, anus, gallbladder, thyroid follicles, ependyma, lymph vessel, skin, sweat gland ducts, mesothelium of body cavities, ovaries, Fallopian tubes, uterus, endometrium, cervix (endocervix), cervix (ectocervix), vagina, labia majora, tubuli recti, rete testis, ductuli efferentes, epididymis, vas deferens, ejaculatory duct, bulbourethral glands, seminal vesicle, oropharynx, larynx, vocal cords, trachea, respiratory bronchioles, cornea, nose, proximal convoluted tubule of kidney, ascending thin limb of kidney, distal convoluted tubule of kidney, collecting duct of kidney, renal pelvis, ureter, urinary bladder, prostatic urethra, membranous urethra, penile urethra, or external urethral orifice, for example.

The cells may be any mammalian cells. The cells may be any human cells. The cells may be selected from the group consisting of lymphocytes, B cells, T cells, cytotoxic T cells, natural killer T cells, regulatory T cells, T helper cells, myeloid cells, granulocytes, basophil granulocytes, eosinophil granulocytes, neutrophil granulocytes, hypersegmented neutrophils, monocytes, macrophages, reticulocytes, platelets, mast cells, thrombocytes, megakaryocytes, dendritic cells, thyroid cells, thyroid epithelial cells, parafollicular cells, parathyroid cells, parathyroid chief cells, oxyphil cells, adrenal cells, chromaffin cells, pineal cells, pinealocytes, glial cells, glioblasts, astrocytes, oligodendrocytes, microglial cells, magnocellular neurosecretory cells, stellate cells, boettcher cells; pituitary cells, gonadotropes, corticotropes, thyrotropes, somatotrope, lactotrophs, pneumocyte, type I pneumocytes, type II pneumocytes, Clara cells; goblet cells, alveolar macrophages, myocardiocytes, pericytes, gastric cells, gastric chief cells, parietal cells, goblet cells, paneth cells, G cells, D cells, ECL cells, I cells, K cells, S cells, enteroendocrine cells, enterochromaffin cells, APUD cell, liver cells, hepatocytes, Kupffer cells, bone cells, osteoblasts, osteocytes, osteoclast, odontoblasts, cementoblasts, ameloblasts, cartilage cells, chondroblasts, chondrocytes, skin cells, hair cells, trichocytes, keratinocytes, melanocytes, nevus cells, muscle cells, myocytes, myoblasts, myotubes, adipocyte, fibroblasts, tendon cells, podocytes, juxtaglomerular cells, intraglomerular mesangial cells, extraglomerular mesangial cells, kidney cells, kidney cells, macula densa cells, spermatozoa, sertoli cells, leydig cells, oocytes, and mixtures thereof. origin.

The cells may also be isolated from a diseased tissue, e.g., a cancer. Accordingly, the cells may be cancer cells. For example, the cells may be isolated or derived from any of the following types of cancers: breast cancer; biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia/lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms' tumor.

The cells may be selected from the group consisting of cord-blood cells, stem cells, embryonic stem cells, adult stem cells, cancer stem cells, progenitor cells, autologous cells, isograft cells, allograft cells, xenograft cells, and genetically engineered cells. The cells may be induced progenitor cells. The cells may be cells isolated from a subject, e.g., a donor subject, which have been transfected with a stem cell associated gene to induce pluripotency in the cells. The stem cell-associated genes may be selected from the group consisting of Oct3, Oct4, Sox1, Sox2, Sox3, Sox15, Klf1, Klf2, Klf4, Klf5, Nanog, Lin28, C-Myc, L-Myc, and N-Myc. The cells may be cells which have been isolated from a subject, transfected with a stem cell associated gene to induce pluripotency, and differentiated along a predetermined cell lineage.

Cells lines of any of the cells disclosed herein may also be used with the methods disclosed herein.

### Transplantation

The invention provides methods of transplanting cells in a subject. The methods typically involve thawing cryopreserved cells in the presence of a polymer, e.g., a polyether, and transplanting the thawed cells in the subject. The method may involve obtaining the cells from a donor that is not the transplant recipient, e.g., for use as an allograft, isograft, or xenograft. The methods may involve obtaining the cells from the subject who is the transplant recipient for use as a autograft. The methods may involve expanding the cells in vitro prior to transplanting. The cells may be cryopreserved while situated in a tissue. The cells may be isolated from a tissue and then cryopreserved. The cells may be cryopreserved while situated in a tissue and isolated from the tissue following thawing.

Cryopreserved cells to be transplanted are thawed in the presence of a polymer, e.g., polyether, at a sufficient concentration for the membranes of the cells to be stabilized and prevent damage to the cells following thawing and during handling and transplantation. The polymer is thought to fix or prevent damage to the cell membranes due to the cryopreservation and/or thawing by associating with the cell membranes. The resulting polymer/cell composition may be further processed before implantation into a subject. For example, the cells may be washed, purified, extracted, expanded, or otherwise treated before implantation into a subject.

The cryopreserved cells may be thawed in the presence of a polymer, e.g., polyether, and seeded in a scaffold material that allows for attachment of cells and facilitates production of an engineered tissue. In one embodiment, the scaffold is formed of synthetic or natural polymers, although other materials such as hydroxyapatite, silicone, and other inorganic materials can be used. The scaffold may be biodegradable or non-degradable. Representative synthetic non-biodegradable polymers include ethylene vinyl acetate and polymethacrylate. Representative biodegradable polymers include polyhydroxyacids such as polylactic acid and polyglycolic acid, polyanhydrides, polyorthoesters, and copolymers thereof. Natural polymers include collagen, hyaluronic acid, and albumin. Hydrogels are also suitable. Other hydrogel materials include calcium alginate and certain other polymers that can form ionic hydrogels that are malleable and can be used to encapsulate cells. Exemplary tissue engineering methods are well known in the art, such as those disclosed in published PCT application WO/2002/016557, U.S. Patent Application Publication 2005/0158358, and U.S. Pat. No. 6,103,255, the contents of which are incorporated herein by reference in their entirety.

The scaffolds are used to produce new tissue, such as vascular tissue, bone, cartilage, fat, muscle, tendons, and ligaments. The scaffold is typically seeded with the cells; the cells are cultured; and then the scaffold implanted. Applications include the repair and/or replacement of organs or tissues, such as blood vessels, cartilage, joint linings, tendons, or ligaments, or the creation of tissue for use as “bulking agents”, which are typically used to block openings or lumens, or to shift adjacent tissue, as in treatment of reflux.

In particular embodiments of the invention, the cells are obtained by performing liposuction on the subject. Accordingly, the inventive system is particularly useful in improving the success of fat transplantation or improving the success of the transplantation of cells derived from fat tissue. In certain embodiments, the cells to be transplanted are harvested from the same person receiving them (i.e., an autologous donation). In certain embodiments, the cells are harvested from the abdomen, thigh, or buttocks of the donor. In certain embodiments, the fat tissue is harvested into a syringe or other container, which may already include the polymer or a composition of the polymer. In certain embodiments, the fat tissue is harvested into a syringe or other container, and cryopreserved in the syringe or other container. The polymer, e.g., polyether, is added to the syringe or other container housing the cryopreserved fat tissue before freezing, before thawing, immediately before thawing, during the thawing, or after thawing. In certain embodiments, the cells to be transplanted are contacted with the polymer during thawing and again immediately before transplantation. For example, the cells may be mixed with the polymer in the operating room or clinic just prior to implantation into a subject. The sterile polymer or composition thereof is mixed with the cells to be transplanted.

After thawing the cells in the presence of the polymer, the cell/polymer composition may be administered to a subject. In certain embodiments, the subject is a human. In certain embodiments, the subject is a mammal. In certain embodiments, the subject is a test animal such as a mouse, rat, rabbit, or dog. The cell/polymer composition is typically administered to a patient in need of a transplant. The cell/polymer composition may be administered to a patient in need of, or desiring, a fat transplant. The subject may be undergoing reconstructive or cosmetic surgery. In certain embodiments, the fat transplantation is used in removing wrinkles. In certain embodiments, fat transplantation is used in soft tissue replacement or augmentation. In certain embodiments, fat transplantation is used in augmentation of the lips, cheeks, breasts, face, buttocks, calves, pectorals, and penis. Typically, autologous fat cells are transplanted back into the donor at a different site from which the cells were taken.

Besides adipocytes, fat tissue has been found to be a source of stem cells (Gimble et al., “Adipose-Derived Stem Cells for Regenerative Medicine” Circulation Research 100:1249-1260, 2007; incorporated herein by reference). Therefore, the inventive system may be useful in stabilizing and preventing damage to stem cells or other cells derived from fat tissue following cryopreservation. In certain embodiments, the inventive system is useful in the transplantation of adult stem cells. In certain embodiments, the inventive system is useful in the transplantation of fibroblasts.

A polymer may be tested for use in transplantation applications by thawing cryopreserved cells in the presence of a test polymer, e.g., a test polyether, and transplanting the resulting composition, comprising thawed cells and the test polymer, into a mouse or other rodent to determine over time the success of the implant. Implants, e.g., fat implants, may be evaluated by various biochemical and pathological measurements, for example, weight of the implant, volume of the implant, assessing markers of apoptosis and/or cell death, assessing mitochondrial ATP levels, or real-time PCR to determine levels of tissue specific markers, e.g., leptin, PPARγ2, or other markers. In certain embodiments, the testing is performed in nude mice. Polymers may also be screened in vitro by thawing cryopreserved cells in the presence of a test polymer, growing the thawed cells in vitro and assaying the cells for markers of apoptosis or cell death, assaying the cells for toxicity, etc. In certain embodiments, the results using a test polymer are compared to the results from a control. In certain embodiments, the control polymer is P188. In certain embodiments, the control polymer is dextran. In certain embodiments, control cells are thawed in the presence of control solution, e.g., normal saline or growth medium.

In the transplantation methods, the polymer may be combined with other biologically active agents and/or pharmaceutically acceptable excipients to form a composition useful for adding to cells to be transplanted. Such agents or excipients may be added during the thawing, e.g., along with the polymer, or following the thawing and prior to transplantation. Such biologically active agents may also work to prevent cell death in a cell or tissue graft, e.g., a fat graft. Excipients may be used to aid in mixing the polymer with the cryopreserved cells to be transplanted or handling and storage of the resulting polymer/cell composition.

Biologically active agents that may be added along with a polymer to the cells to be transplanted include, but are not limited to, antioxidants, vitamins, membrane stabilizers, minerals, osmotic protectants, coenzymes, viscosity enhancers, hormones, and growth factors. Numerous mechanisms have been implicated in the cause of cell death in transplanted cells, for example, membrane disruption and free radical formation. Antioxidants may be used to reduce free radical formation. Antioxidants scavenge free radicals and prevent damage caused by reactive oxygen species. In certain embodiments, a polymer/cell composition further comprises an antioxidant. The polymer and antioxidant are thought to improve viability of cryopreserved cells post-thaw and thereby improve transplantation results. The antioxidants may be enzymatic or nonenzymatic antioxidants. Enzymatic antioxidants include, for example, superoxide dismutase, glutathione peroxidase, and catalase. Exemplary non-enzymatic antioxidants include ascorbic acid (vitamin C), alpha-tocopherol (vitamin E), vitamin A, glutathione, carotenoids (e.g., lycoprene, lutein, polyphenols, β-carotene), flavonoids, flavones, flavonols, glutathione, N-acetyl cysteine, cysteine, lipoic acid, ubiquinal (coenzyme Q), ubiquinone (coenzyme Q10), melatonin, lycophene, butylated hydroxyanisole, butylated hydroxytoluene (BHT), benzoates, methyl paraben, propyl paraben, proanthocyanidins, mannitol, and ethylenediamine tetraacetic acid (EDTA). In certain embodiments, the antioxidant is a metallic antioxidant. In certain embodiments, the antioxidant is selenium. In certain embodiments, the antioxidant is zinc. In certain embodiments, the antioxidant is copper. In certain embodiments, the antioxidant is germanium.

In certain embodiments, a polymer/cell composition further comprises a vitamin. The vitamin may be an antioxidant. In certain embodiments, the vitamin is alpha-tocopherol (vitamin E). In certain embodiments, the vitamin is ascorbic acid (vitamin C). In certain embodiments, the vitamin is coenzyme Q10. In certain embodiments, the vitamin is beta-carotene. Other vitamins that may be added to the inventive polymer/cell composition include vitamin A, vitamin B₁ (thiamine), vitamin B₂ (riboflavin), vitamin B₃ (niacin), vitamin B₄ (adenine), vitamin B₅ (pantothenic acid), vitamin B₆ (pyridoxine), vitamin B₇ (biotin), vitamin B₉ (folic acid), vitamin B₁₂ (cyanocobalamin), vitamin D (ergocalciferol), and vitamin K.

In certain embodiments, a polymer/cell composition further comprises another membrane stabilizer besides the polymer used during the thawing described herein. In certain embodiments, the membrane stabilizer is a second polymer. The membrane stabilizer is thought to further facilitate the sealing of cell membranes to prevent cellular injury. In certain embodiments, the membrane stabilizer is polyethylene glycol. Different molecular weight PEGs and different isomers of PEG may be used. In certain embodiments, copolymers of PEG are used in the cell/polymer compositions.

In certain embodiments, a polymer/cell composition further comprises an osmotic protectant. Such an osmotic protectant may aid in protecting the cells in the cell/polymer composition from osmotic damage or osmotic stress. In certain embodiments, the osmotic protectant is a polysaccharide. In certain embodiments, the osmotic protectant is maltose. In certain embodiments, the osmotic protectant is raffinose. In certain embodiments, the osmotic protectant is sucrose. In certain embodiments, the osmotic protectant is mannitol. In certain embodiments, the osmotic protectant is PEG.

In certain embodiments, a polymer/cell composition further comprises a viscosity enhancer. In certain embodiments, the viscosity enhancer is a polymer. In certain embodiments, the viscosity enhancer is a polysaccharide. In certain embodiments, the viscosity enhancer is cellulose or a cellulose derivative. In certain embodiments, the viscosity enhancer is carboxymethylcellulose. In certain embodiments, the viscosity enhancer is methyl cellulose. In certain embodiments, the viscosity enhancer is ethyl cellulose, hydroxyethylcellulose, hydroxypropylcellulose, hydroxyethyl ethylcellulose, hydroxyethylcellulose, hydroxypropylcellulose, or hydroxybutyl cellulose. Other exemplary viscosity enhancers include synthetic polymers (e.g., acrylamides, acrylates). In certain embodiments, the viscosity enhancer is a wax or fatty alcohol (e.g., cetyl alcohol).

In certain embodiments, a polymer/cell composition further comprises an alcohol (e.g., polyphenols, fatty alcohol). In certain embodiments, a polymer/cell composition further comprises a hormone or growth factor. In certain embodiments, the hormone or growth factor is insulin, glitazones, cholesterol, VEGF, FGF, EGF, PDGF, etc. In certain embodiments, the polymer/cell composition further comprises an organic acid (e.g., lipoic acid). In certain embodiments, the polymer/cell composition further comprises a small organic molecule (e.g., anthocyanins, capsaicins). In certain embodiments, the polymer/cell composition further comprises a steroidal compound (e.g., cholesterol). In certain embodiments, the polymer/cell composition further comprises a lipid.

In certain embodiments, cryopreserved cells, e.g., fat cells, are combined with P188 and vitamin C for transplantation into a subject. In certain embodiments, cryopreserved cells, e.g., fat cells, are combined with P188 and glutathione. In certain embodiments, cryopreserved cells, e.g., fat cells, are combined with P188 and lipoic acid. In certain embodiments, cryopreserved cells, e.g., fat cells, are combined with P188 and vitamin E.

The formulations of the polymers described herein may be prepared by any method known or hereafter developed in the art of pharmaceuticals. In general, such preparatory methods include the step of bringing the polymer into association with one or more excipients and/or one or more other biologically active agents. The relative amounts of the polymer, the pharmaceutically acceptable excipient(s), and/or any additional agents in a composition of the invention will vary, depending upon the identity of the polymer, size of the polymer, implantation site, and/or subject. By way of example, the composition to be mixed with cryopreserved cells, e.g., during the thawing, to be transplanted may comprise between 1% and 99% (w/w) of the polymer.

Formulations of the polymer may comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular formulation desired. Remington's The Science and Practice of Pharmacy, 21^{st} Edition, A. R. Gennaro, (Lippincott, Williams & Wilkins, Baltimore, Md., 2006; incorporated herein by reference) discloses various excipients used in formulating pharmaceutical compositions and known techniques for the preparation thereof. Except insofar as any conventional excipient is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, the pharmaceutically acceptable excipient is at least 95%, 96%, 97%, 98%, 99%, or 100% pure. In some embodiments, the excipient is approved for use in humans and for veterinary use. In some embodiments, the excipient is approved for use in humans by the United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of the polymer compositions include, but are not limited to, inert diluents, dispersing agents, surface active agents and/or emulsifiers, disintegrating agents, preservatives, buffering agents, lubricating agents, and/or oils. Such excipients may optionally be included in the inventive formulations. Excipients such as coloring agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween®20], polyoxyethylene sorbitan [Tween®60], polyoxyethylene sorbitan monooleate [Tween®80], sorbitan monopalmitate [Span®40], sorbitan monostearate [Span®60], sorbitan tristearate [Span®65], glyceryl monooleate, sorbitan monooleate [Span®80]), polyoxyethylene esters (e.g. polyoxyethylene monostearate [Myrj®45], polyoxyethylene hydrogenated castor oil, polyethoxylated castor oil, polyoxymethylene stearate, and Solutol), sucrose fatty acid esters, polyethylene glycol fatty acid esters (e.g. Cremophor®), polyoxyethylene ethers, (e.g. polyoxyethylene lauryl ether [Brij®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, cetrimonium bromide, cetylpyridinium chloride, benzalkonium chloride, docusate sodium, etc. and/or combinations thereof.

Exemplary preservatives may include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include, but are not limited to, ethanol, polyethylene glycol, phenol, phenolic compounds, bisphenol, chlorobutanol, hydroxybenzoate, and phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus®, Phenonip®, methylparaben, Germall 115, Germaben II, Neolone™, Kathon™, and Euxyl®. In certain embodiments, the preservative is an antioxidant. In other embodiments, the preservative is a chelating agent.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

### Other Uses

The cryopreserved cells may be used for any appropriate downstream application, e.g., research, drug discovery, biologics production, etc. The cells may be used for microscopy, e.g., in combination with immunostaining, in situ hybridization, etc. The cells may be used for functional studies such as gene knockdown or overexpression studies. The cells may be to study various molecular pathways, e.g., cell cycle, cell signaling, gene regulatory, etc. The cells may be separated by flow cytometry. The cells may be used to create cell lines. The cells may be used for fractionation studies, e.g., to purify proteins or molecules from different cellular compartments. The cells may be used for studying different disease pathways, e.g., cancer. The cells may be transplanted into an animal model, e.g., to study tumor growth. The cells may be used for gene, e.g., mRNA or miRNA, profiling studies. The karyotype or genotype of the cells may be evaluated. The cells be may used for isolation of various biomolecules, e.g., antibodies, proteins, RNA, DNA, ligands, etc.

The cells may be used for automated microscopy for high-content screening, e.g., for lead identification and compound characterization. The cells may be used for the evaluation, e.g., by screening, e.g., high-throughput screening, of compounds, e.g., small-molecules, siRNAs, peptides, etc., for a desired activity, e.g., inhibition of cell growth, modulation of a particular biochemical pathway, modulation of the expression of a certain gene, binding to a target, etc.

The cells may be used in a biopharmaceutical context for the production and isolation of therapeutic molecules, e.g., antibodies, enzymes, etc. The cells may be shipped, e.g., on dry ice in the presence of a polymer, e.g., a polyether, to a customer, collaborator, etc. The cells may be evaluated for contamination, e.g., bacterial, mycoplasmal, viral, etc. The uses disclosed herein are not intended to be limiting and variety of other uses for the cryopreserved cells are also envisioned and will be apparent to the skilled artisan.

### Kits

The invention also provides packages or kits, comprising one or more polymers, e.g., polyethers, or polymer components as described herein in a container. For example, the container may include a polyether or composition of a polyether ready for use in thawing cryopreserved cells. Instructions for the use of the polymer may also be included. In particular, the instructions may include information regarding the contacting of the polymer with cryopreserved cells during thawing of the cells. Such instructions may also include information relating to administration of a polymer/cell composition to a patient, e.g., following thawing of the cells in the presence of the polymer. The package may also include one or more containers containing biologically active agent(s) to be included in the polymer/cell composition prior to administration. The package can also include a notice associated with the container, typically in a form prescribed by a government agency regulating the manufacture, use, or sale of medical devices and/or pharmaceuticals, whereby the notice is reflective of approval by the agency of the compositions, for human or veterinary administration in tissue transplantation.

The package may include a device or receptacle for preparation of the polymer/cell composition. The device may be, e.g., a measuring or mixing device.

The package may also optionally include a device for administering a polymer/cell composition of the invention. Exemplary devices include specialized syringes, needles, and catheters that are compatible with a variety of laryngoscope designs.

The components of the kit may be provided in a single larger container, e.g., a plastic or styrofoam box, in relatively close confinement. Typically, the kit is conveniently packaged for use by a health care professional. In certain embodiments, the components of the kit are sterilely packaged for use in a sterile environment such as an operating room or physician's office.

### EXAMPLES

### Example 1

### An Agent for Improved Cryopreservation of Adipose Tissue

Background: In a study of adipocyte resuscitation using a tri-block copolymer (P188) we have discovered a significant improvement in graft preservation. We hypothesized that a similar strategy may be utilized to protect frozen fat as well. In this study cryo-banked adipose tissue was treated with various agents as a protectant followed by injection into a nude mouse model and serial explantation and analysis.

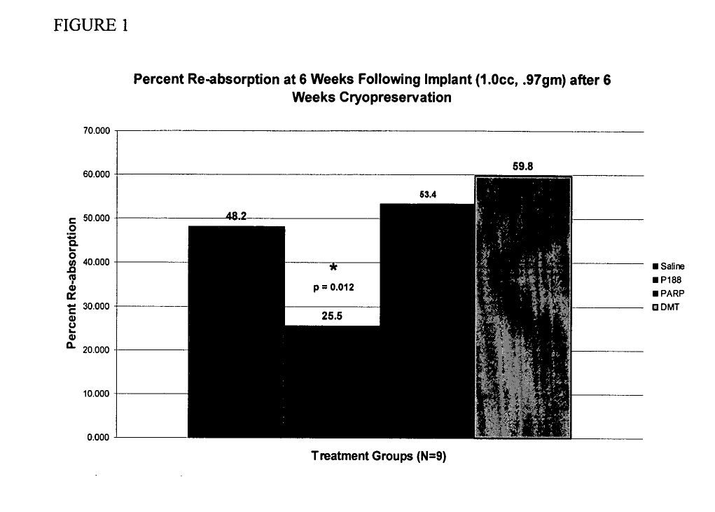

Methods: Fat was obtained via human liposuction aspirates, washed with saline and centrifuged. Aliquots of fat were treated with one of four agents: polymer (P188), PARPi (anti-apoptosis control), DMSO+Trehalose (gold standard), or saline as a negative control. The four non-DMSO containing groups were snap frozen and stored at −80° C. for six weeks, the DMSO group was slow cooled at −20° C. (24 hrs) then stored at −80° C. for six weeks. Thawed samples where then implanted into nude mice (1.0 cc and 0.97 g weight). Samples were serially harvested at 3, 6, and 9 days and at 6 weeks. The explanted fat nodules were weighed and analyzed for G3PH activity, ATP levels, cell counts, and apoptotic activity. (FIG. 1)

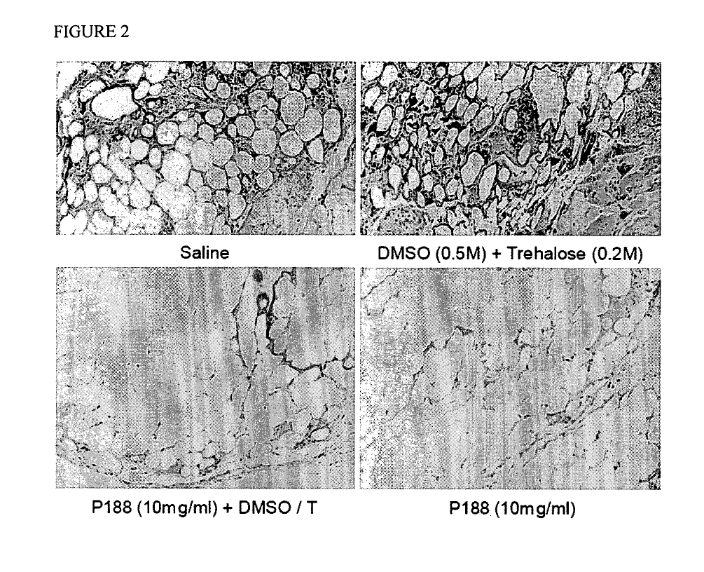

Results: During the first 9 days there was neither a statistical difference between any of the groups with implant weight nor apoptotic activity. However at 6 weeks the DMSO+Trehalose controls exhibited up to 60% re-absorption. PARPi demonstrated a similar 53% resorption (p=0.004). Significantly, grafts treated with P188 demonstrated only 25% resorption (p=0.012) at 6 weeks. The ATP levels at 6 weeks were higher in P188 treated grafts when compared to saline controls. However, there where no significant differences in ATP levels between P188 and DMSO+Trehalose at 6 weeks. Histological examination demonstrated superior adipose tissue structure in the P188 treated samples versus the other groups. (FIG. 2) Interestingly, the DMSO+Trehalose samples histologically contained large amounts of fibrotic tissue and large vacuolated spaces.

Conclusions: Treatment of cryopreserved cells with a membrane stabilizing agent P188 provides a method for cryoperservation of fat without the toxic effects of DMSO. These results indicate that the polymer is a viable agent for a use with clinically banked adipose tissue aspirates.

### Example 2

### Viability of Transplanted Cryopreserved Cells Treated with a Polyether During the Thawing Process

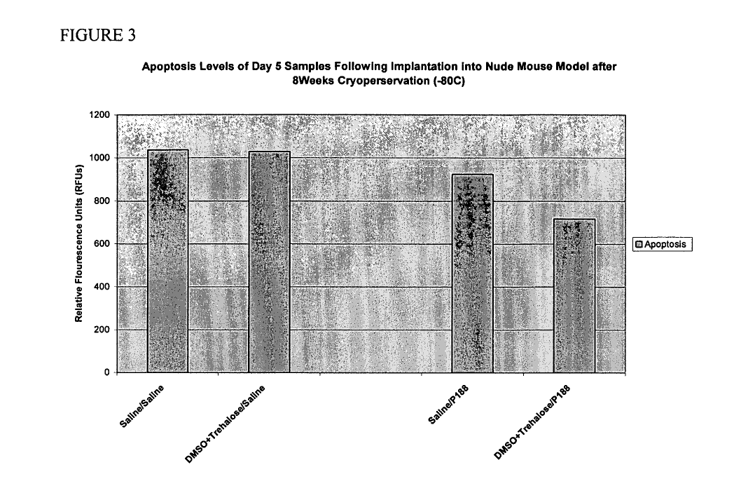

The effectiveness of P188 used during the thawing process to reduce the amount of cell death (apoptosis) was evaluated. See FIG. 3. Samples were treated with either saline (control) or DMSO+Trehalose (gold standard) and then frozen at −80° C. for eight weeks. Samples were then either thawed in saline or thawed in P188 solution. After thawing the each group was injected in 1.0 cc aliquots into a nude mouse model. On day 5 injections were sampled from each group and the amount of cell death in the graft was measured using fluorescent labels. A comparison of P188 treated groups to the saline treated groups, indicates reductions in the amount of cell death when P188 is used during the thawing process. These results indicate that P188 improves outcomes by targeting injury during the thawing period irrespective of the use of a prior cryopreservative.

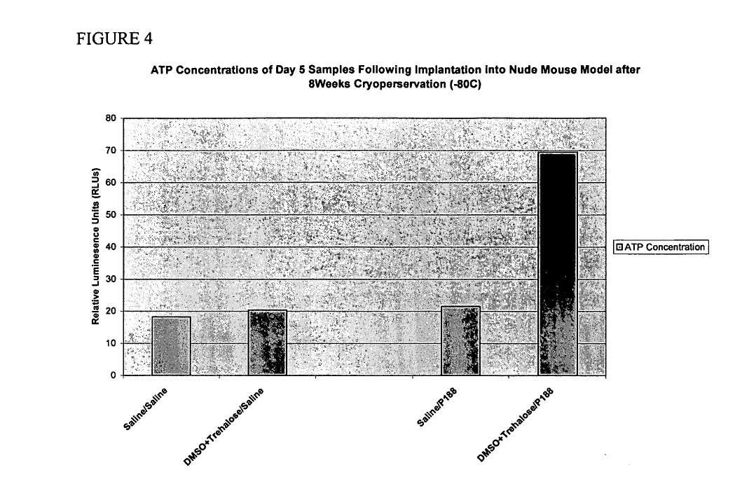

The functional improvements in fat grafts when P188 is used during the thawing process were also evaluated. (FIG. 4) These samples were treated with either saline (control) or DMSO+Trehalose (gold standard) and then frozen at −80° C. for eight weeks. Samples were then either thawed in saline or thawed in P188 solution. After thawing each group was injected in 1.0 ml aliquots into a nude mouse model. On day 5 injections were sampled from each group and the amount of ATP was measured.

A comparison of P188 treated groups to the saline treated groups, indicates an increase in ATP levels when P188 is used during the thawing process. DMSO+Trehalose without P188 treatment demonstrated slightly higher ATP levels than saline which was expected. When the gold standard is then treated with P188, during thawing, graft ATP levels are dramatically higher. Also the saline treated group when thawed in P188 demonstrated slightly improved ATP levels. These results indicate that P188 increases cellular function by protecting cells from membrane injury during the thaw process, regardless of use of prior cryopreservative. Thus, when P188 is used in the thaw it improves graft function.

### Example 3

### Protocol for Fat Cryopreservation, Thawing and Transplantation

Fat is first isolated from a subject using liposuction. The fat is dispensed into aliquots of about 30 ml in syringes, e.g., 60 ml syringes. A cryoprotectant is optionally added to the aliquots. The fat aliquots are then frozen at −80° C. The fat aliquots are stored for later use. Just prior to thawing an equal volume of a polymer, e.g., polyether, typically P188, solution is added to the cryopreserved fat aliquot. The cryopreserved fat aliquot is then thawed in the presence of the polymer by incubation in a water bath at about 37.5° C. for about 20 min, followed by further incubation on gentle rocker for about 15 min at about 37.5° C. The sample is then spun and transplanted into a subject.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise. In addition, the invention encompasses compositions made according to any of the methods for preparing compositions disclosed herein.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

1. A method for improving the viability of cryopreserved cells, the method comprising:
thawing cryopreserved cells in the presence of a polyether.

2. (canceled)

3. The method of claim 1, wherein the polyether is a tri-block co-polymer.

4-13. (canceled)

14. The method of claim 1, wherein the polyether is a tri-block co-polymer of polyethylene glycol and polypropylene glycol.

15. (canceled)

16. The method of claim 1, wherein the polyether is POLOXAMER P188 or POLOXAMER P108.

17. (canceled)

18. The method of claim 1, wherein the polyether is polyethylene glycol, polysorbate 80, meroxapol, or poloxamine.

19-21. (canceled)

22. The method of claim 1, wherein the polyether is at least 95% pure, at least 98% pure, or at least 99% pure.

23-24. (canceled)

25. The method of claim 1, wherein the molecular weight of the polyether ranges from approximately 1,000 g/mol to approximately 10,000 g/mol.

26-28. (canceled)

29. The method of claim 1, wherein the polyether is non-ionic.

30. The method of claim 1, wherein the polyether is added to the cryopreserved cells before thawing.

31. The method of claim 1, wherein the polyether is added to the cryopreserved cells immediately before thawing.

32. The method of claim 1, wherein the polyether is added to the cryopreserved cells after thawing has begun.

33. The method of claim 1, wherein the concentration of the polyether ranges from about 1 mg/ml to about 10 mg/ml.

34-52. (canceled)

53. The method of claim 1 further comprising washing the cells.

54. (canceled)

55. The method of claim 1, wherein the cells are cryopreserved in the presence of one or more agents selected from the group consisting of dimethyl sulfoxide, ethylene glycol, glycerol, propylene, glycol, trehalose, dextrose, sucrose, glucose, maltose, and serum.

56. The method of claim 1, wherein the cryopreserved cells are selected from the group consisting of cord-blood cells, stem cells, embryonic stem cells, adult stem cells, progenitor cells, autologous cells, allograft cells, xenograft cells, and genetically engineered cells.

57. The method of claim 1, wherein the cryopreserved cells are cells of a tissue selected from the group consisting of: connective, nervous, muscle, and epithelial.

58. The method of claim 57, wherein the connective tissue is adipose tissue.

59. (canceled)

60. The method of claim 1, wherein the cryopreserved cells are selected from the group consisting of lymphocytes, B cells, T cells, cytotoxic T cells, natural killer T cells, regulatory T cells, T helper cells, myeloid cells, granulocytes, basophil granulocytes, eosinophil granulocytes, neutrophil granulocytes, hypersegmented neutrophils, monocytes, macrophages, reticulocytes, platelets, mast cells, thrombocytes, megakaryocytes, dendritic cells, thyroid cells, thyroid epithelial cells, parafollicular cells, parathyroid cells, parathyroid chief cells, oxyphil cells, adrenal cells, chromaffin cells, pineal cells, pinealocytes, glial cells, glioblasts, astrocytes, oligodendrocytes, microglial cells, magnocellular neurosecretory cells, stellate cells, boettcher cells; pituitary cells, gonadotropes, corticotropes, thyrotropes, somatotrope, lactotrophs, pneumocyte, type I pneumocytes, type II pneumocytes, Clara cells; goblet cells, alveolar macrophages, myocardiocytes, pericytes, gastric cells, gastric chief cells, parietal cells, goblet cells, paneth cells, G cells, D cells, ECL cells, I cells, K cells, S cells, enteroendocrine cells, enterochromaffin cells, APUD cell, liver cells, hepatocytes, Kupffer cells, bone cells, osteoblasts, osteocytes, osteoclast, odontoblasts, cementoblasts, ameloblasts, cartilage cells, chondroblasts, chondrocytes, skin cells, hair cells, trichocytes, keratinocytes, melanocytes, nevus cells, muscle cells, myocytes, myoblasts, myotubes, adipocyte, fibroblasts, tendon cells, podocytes, juxtaglomerular cells, intraglomerular mesangial cells, extraglomerular mesangial cells, kidney cells, kidney cells, macula densa cells, spermatozoa, sertoli cells, leydig cells, oocytes, and mixtures thereof.

61-74. (canceled)

75. A method for improving the viability of cryopreserved cells, the method comprising:
freezing cells in the presence of a cryoprotectant; and
thawing cryopreserved cells in the presence of a polyether.

76-78. (canceled)

79. The method of claim 75 further comprising the step of transplanting the cells into a subject.
